(19) United States
(12) Patent Application Publication
(10) Pub. No.: US 2012/0129193 A1
(43) Pub. Date: May 24, 2012

(54) DEVICE AND METHOD FOR ANALYSING CELLS

(75) Inventors: Christian Hennig, Hannover (DE); Gesine Hansen, Hannover (DE)

(73) Assignee: MEDIZINISCHE HOCHSCHULE HANNOVER, Hannover (DE)

(21) Appl. No.: 13/126,116

(22) PCT Filed: Oct. 27, 2009
(86) PCT No.: PCT/EP09/64163

§ 371 Date: Jul. 18, 2011

(30) Foreign Application Priority Data
Oct. 27, 2008 (DE) DE 102008053270.3

(51) Int. Cl. G01N 21/64; C12M 1/34

(52) U.S. Cl. 435/7.24; 435/7.1; 435/287.2; 435/7.21

(57) ABSTRACT

There is provided a carrier substrate for nonspecific immobilization of living bacterial and/or eukaryotic cells, especially of animal cells, which can be present as single cells, cell agglomerates or tissue sections. The surface of the carrier substrate is provided at least sectionally with a layer having or consisting of oligonucleic acids, preferably having ribonucleic acids covalently coupled to the carrier substrate, e.g. RNA, preferably single-stranded or double-stranded DNA.

The invention relates to a device and its use in a method for the analysis of immobilized cells, in particular of living prokaryotic and eukaryotic cells, particularly preferred animal and human cells. According to the invention a device with an at least partially optically translucent channel is provided, which is to be arranged in a beam path of an optical detection device, e.g. of an optical microscope and/or of an optical scanning device with a laser beam (laser scanner), wherein an at least sectional surface coating of the channel allows the effective, non-cell type specific or non-cell selective adsorption or immobilization of living cells, e.g. of a culture, of medical samples or of biopsies. The effective immobilization of cells, independent from the cell-type on at least a section of the surface of the channel allows the contacting of the immobilized cells with dye-coupled probes, also called detection conjugate, in particular dye-coupled antibodies, the optical detection of the dye, the inactivation of the dye, and the repeated contacting of the immobilized cells with a further detection conjugate for successive detection of different analytes, in particular of surface-bound and/or cell-internal antigens by the antigen-specific binding portion of the detection conjugate.

### STATE OF THE ART

Mahnke and Roederer (Clin Lab Med. 2007, 27(3) (2007)) describe, how for flow cytometry a majority of antibody conjugates can be coordinated to one another for the detection of antigens on cells, in order to avoid interferences of the fluorochrome portions at their simultaneous application, each having differing emission spectra. For separation of overlapping emission spectra, in particular with nonspecific excitation of a fluorochrome by energy transfer from another fluorochrome, a computer-aided evaluation of the signals is proposed, in which nonspecific emission signals can be detracted as background.

Laffers et al. (Cytometry Part A 69A: 127-130 (2006)) describe the analysis of living cells by FACS or microscopy of lysed denatured cells, which subsequent to incubation in a suspension with a labelled antibody were applied onto a microscope slide and immobilized by drying. The antibody-fluorochrome conjugates each contained different fluorochromes. For evaluation the data from flow cytometry (FACS) or from microscopy using fluorescence detection were superimposed in a computer-aided way.

Támok (Cytometry Part A 69 A:555-562 (2006)) in addition to cytometry using antibody conjugates with different fluorochromes mentions the successive application of antibody-fluorochrome conjugates, the bleaching of fluorochromes by irradiating, the activation and the destruction of fluorochromes by radiation.

Perfetto et al. (Nature 648-654 (2004)) describe flow cytometers for the detection of a multitude of fluorochromes by specific excitation and wavelength specific detection of emitted light each in rapid succession, which is called simultaneous measuring. It is shown that different fluorochromes which are concurrently present in a sample nonetheless show nonspecific emissions upon excitation with a wavelength which is fluorochrome-specific, which is attributed to an energy transfer between the fluorochromes. For avoidance of nonspecific signals the coordination of the concurrently utilized antibody-fluorochrome conjugates in an empirical method is proposed. For further reduction of nonspecific emission signals a mathematical compensation is proposed with which emissions are substracted which can be traced back to the nonspecific energy transfer between fluorochromes.

U.S. Pat. No. 6,150,173 describes a computer-controlled pipetting automat by which antibody-fluorochrome conjugates are successively incubated with a sample, after irradiation with an excitation wavelength the emitted radiation is measured, subsequently the fluorochrome is destructed by irradiation with UV, and a new antibody-fluorochrome conjugate is added to the same sample. As a sample, cells immobilized on a microscope slide are used by drying, fixation by acetone and renewed drying.

DE 197 09 348 and WO 2007/101706 A1 describe that prior to detection of a specific antibody-fluorochrome conjugate the biological sample is treated by irradiation in order to reduce undesired fluorescence.

WO 2007/024701 describes a flow chamber for microscopic observation, within which half-shell shaped elevations cover a part of the cross-sectional area and serve as bow-nets for intake of micro-particles or cells. The agglomeration of nanoparticles makes these adhere to the bow-nets, so that the agglomeration is used as an optical indicator.

US 2008/01 38848 A1 describes the specific shape of a flow cell in which for example yeast cells can be kept stationarily in the current and can be analyzed microscopically.

WO 2007/106598 describes the coating of surfaces by specific binding molecules, for example antibodies, in order to establish an interaction with an analyte, and also mentions nucleic acids as specific binding molecules for analytes in addition to antibodies, without experimentally demonstrating the suitability of nucleic acids for specific immobilization. The specificity of the analytical method is based on the unique specific binding of an analyte from the sample so that for the detection the presence of a binding of the analyte to the surface is detected only; a further specific detection of components of bound analytes does not take place. This is because the individual cell-types are bound to the surface by the cell-type specific binding, which is caused by the nucleic acids. Therefore, the type-specifically immobilized cells already represent a sub-group selected from the mixture of cells utilized, such that it is sufficient for the analysis to detect the presence of the highly specifically selected immobilized cells. Furthermore, due to the specific selection a more extensive analysis would be limited to the immobilized sub-group.

US 2002/0128234 A1 uses derivatized PEG for bonding of the binding molecule.

The devices known for immobilization of cells are disadvantageous in that either only denatured cells can be fixed on a carrier substrate, or only predetermined cells can be immobilized specifically, for example by antibodies bound to the carrier substrate. The practice-oriented methods for analysis of cells each use flow-cytometric detection of specifically labelled cells, so that necessarily all specific labelling reagents have to be optically active and have to be in contact with the cells at the same time, whereby interfering interactions of the optically detectable moieties result.

### OBJECT OF THE INVENTION

In view of the known state of the art, it is the object of the present invention to provide a device and a method for the analysis of cells, wherein the cells, in particular bacterial and animal cells, can be immobilized alive on a carrier substrate in a simple way.

### GENERAL DESCRIPTION OF THE INVENTION

The invention achieves the object by the features of the claims, especially by providing a carrier substrate for use for the nonspecific immobilization of living bacterial and/or eukaryotic cells, in particular animal cells, which can be present as individual cells, cell agglomerates or tissue sections, in particular the use of the carrier substrate in an analytical method and the use of the carrier substrate in a device for analysis. In a first embodiment the surface of the carrier substrate is provided with a layer having oligonucleic acids or consisting of oligonucleic acids, preferably with ribonucleic acids, for example RNA, coupled to the carrier material covalently and/or by means of coupling groups with one of their ends (3′ or 5′), preferably single-stranded or double-stranded DNA at least in a section between the spacers. For the purposes of the invention the term oligonucleic acids comprises natural and synthetic nucleic acid chains, single-stranded or double-stranded each, preferably with a length of at least 5 nt (nucleotides), more preferred 10 to 10.000 nt, even more preferred up to 5.000 nt or up to 1.000 nt, particularly preferred 10 to 100 or up to 50 nt. Preferably, the at least sectional coating of the carrier substrate consists of chemically bound single-stranded and/or double-stranded oligonucleic acids, in particular DNA. According to the invention the oligonucleic acids have a nucleotide sequence which in solution assumes an essentially linear conformation, e.g. under cell physiological conditions. Therefore, suitable nucleic acids comprise or consist of a sequence of at least 90%, preferably 99% nucleotides, which each comprise the same of A (adenine), T (thymine), G (guanine), C (cytosine), I (inosine) or U (uridine) or consist of their nucleotides, e.g. oligo-A, oligo-dA, oligo-T, oligo-dT, oligo-C, oligo-dC, oligo-G, oligo-dG, oligo-I, oligo-dI, oligo-U, oligo-dU with less than 10%, preferably less than 1% of the total number of nucleotides being other nucleotides, particularly preferred of a sequence of nucleotides having the same base respectively, e.g. of nucleotides, each containing A or T, particularly preferred consist only of a sequence of one of the nucleotides A or dA and of T or dT, respectively, and e.g. form a homooligomer. Preferably, the afore-mentioned oligonucleic acids are single-stranded.

In case the oligonucleic acids have a double-stranded conformation, they comprise or consist of adjacent sections, a first section of which consists of oligo-A and/or oligo-dA, and a second section of oligo-T and/or oligo-dT. Preferably, the first and second section each comprise less than 10%, preferably less than 1% of other nucleotides of the total number of nucleotides, and preferably consist of a sequence of only the same nucleotide each. Preferably, sections consist of 3 to 100, preferably 5 to 50 nucleotides. Between the first and second sections, a maximum of 10, preferably a maximum of 2 to 5, particularly preferred no other nucleotide is arranged. The first and second sections can be arranged on a common nucleotide strand, or on 2 complementary hybridized strands. Preferably, the first and second sections have the same number of nucleotides, particularly preferred a double-stranded oligonucleotide consists of a sequence of first and complementary second sections, which have the same number of nucleotides each, and are arranged symmetrically to the central nucleotide or the central nucleotides of the oligonucleotide, particularly of a first section and a complementary second section.

It was found that oligonucleic acids are particularly suitable for use for the immobilization of living animal cells under physiological conditions when the oligonucleic acids have a linear or a filamentary prolate conformation, and therefore essentially have e.g. no three-dimensional folding correspondingly, which e.g. occurs for aptamers. Preferably, the oligonucleic acids of linear conformation are bound to the carrier substrate at one of their ends, whereas the other end is uncombined. It is currently assumed that it is due to the combination of their linear conformation with only one of their ends forming of a bond to the carrier substrate in aqueous medium that the oligonucleic acids are arranged approximately in perpendicular and filamentary to the carrier substrate. The bond of the one end of the oligonucleic acids to the carrier substrate is preferably covalent, e.g. via at least divalent covalently binding coupling groups, alternatively by means of coupling groups, of which e.g. a first binding partner is connected to the carrier substrate and a second binding partner specifically binding to the first one is bound to an oligonucleic acid. A preferred combination of first and second binding partners is avidin or streptavidin with biotin. As a coupling group, a nucleotide can be terminally arranged at the oligonucleic acid, too, which is connected to the carrier substrate, e.g. covalently by means of intermediate groups or with additional first and second binding partners. A nucleotide arranged at the oligonucleic acid which is connected to the carrier substrate can optionally carry a second oligonucleic acid of the invention such that two or more oligonucleic acids can be bound to one coupling group, e.g. dependent from the coupling group.

It was found that cells are immobilized nonspecifically in that section of the carrier substrate in which according to the invention oligonucleic acids having a linear conformation are bound by one of their ends.

Preferably, the carrier substrate is silicate-containing, for example silicate-containing glass, particularly preferred borosilicate glass.

Preferably, the oligonucleic acids according to the invention are bound with a number of at least 5,000/μm², preferably at least 7,000/μm², even more preferred at least 10,000/μm² carrier substrate. This is because it was found that the cell-type unspecific immobilization of cells is dependent from the number of oligonucleic acids per unit of area of the carrier substrate, and that it is more effective with increasing number, or the binding of cells does not suffice below this number per unit of area for the immobilization according to the invention independent from the cell-type, or does not take place efficiently.

Optionally in addition or as an alternative to the at least sectional coating of the carrier substrate by bound oligonucleic acids within the channel the carrier substrate has a frame which is arranged on the carrier substrate as a form-fitting projection to the sample, for example a tissue section. By means of such a frame on the carrier substrate the cells which are contained in a tissue section can be immobilized form-fittingly and positionally accurately on the carrier substrate, so that living cells within the tissue section can be analyzed repeatedly, wherein individual sections of the tissue section can each be optically analyzed repeatedly positionally accurately, even if the carrier substrate is removed between individual steps of detection from the visual field of the microscope that is used for the analysis. This is because both the form-fitting mounting of a tissue section on the carrier substrate and the immobilization of cells on the surface of the carrier substrate coated with oligonucleic acids takes place independently from the cell-type and therefore not cell-selectively, and positionally accurately, so that the positions of immobilized cells in the embodiments of the invention in relation to the carrier substrate are positionally accurate and are therefore analyzable repeatedly. Correspondingly, the analytical method using the silicate glass coated with covalently bound oligonucleic acids that is used as a carrier substrate is preferably cyclic, whereby in each cycle, which at least contains or consists of the steps of contacting with antibody conjugate, detecting of its fluorochrome portion, and inactivating the fluorochrome portion, a detection conjugate having a different antigen specificity, in particular an antibody conjugate is used and the cells are immobilized alive or are fixated denatured or perforated.

Instead of an antibody as in antibody conjugates, detection conjugates can have a different binding group than an antibody which is coupled to a fluorochrome. Correspondingly, the term of the antibody conjugates for the purposes of the invention also comprises fluorochrome-coupled binding molecules which are specific for a component of a cell. Such binding molecules can be selected from synthetic single-chain or multi-chain antibodies, aptameres (oligonucleic acids which specifically bind to an epitope), lectins and receptor-specific binding molecules.

Therein, the coating of silicate glass by oligonucleic acids having a linear conformation in an aqueous medium according to the invention has the advantage of generating a high density and uniform distribution of immobilized living cells at contact with suspended cells. Additional reagents are not necessary for the immobilization of prokaryotic and/or eukaryotic cells on the silicate glass surface coated with covalently bound oligonucleic acids such that the method for immobilization can consist of the contacting of the silicate glass surface coated with oligonucleic acids with cells or cell agglomerates or tissue sections in suspension, wherein the suspension can be aqueous with a salinity compatible to the cells, in particular a cell-physiological medium, optionally without organic components.

A particular advantage of the immobilization according to the invention of living eukaryotic, in particular of animal and of human cells on a carrier substrate over known immobilization methods is that for the oligonucleotide coating according to the invention a non-cell-type specific immobilization takes place, but rather a non-specific immobilization of prokaryotic cells and of eukaryotic cells, e.g. of bacteria and particularly of animal cells including human cells. The coating according to the invention allows the immobilization of animal cells over a long period of time, for example at least 4 h, preferably at least 8 to 10 h, wherein the cells are viable in cell culture medium within the channel which is formed by the carrier substrate, the spaced lid and two spacers. The spacers extend over the spacing between the carrier substrate and the lid, wherein the channel formed between carrier substrate, lid and spacers has openings for inlet and outlet of liquid compositions at one, preferably at two spaced positions, preferably at a first end and an opposite second end. The cross-section of the channel can be chosen freely, preferably the carrier substrate and the opposite spaced-apart lid are parallel to each other in the overlapping area. The spacers which cover the distance between carrier substrate and lid while forming at least one of an inlet opening and an outlet opening, preferably one inlet and one outlet opening each, can be mounted onto the carrier substrate and/or the lid or be formed integrally with one of these, so that one or two openings, through which the inner volume of the channel is accessible, is arranged in the lid and/or in the carrier substrate. The carrier substrate preferably is optically transparent and in the form of a plate, alternatively in the form of particles. Particularly preferred, the lid is optically transparent as well.

Furthermore it has been found that the use of the carrier substrate that is provided with bound oligonucleic acids according to the invention in the analytical method effectuates that immobilized animal cells essentially maintain their three-dimensional elliptical form or spherical form of the suspended state so that these immobilized cells show an enhanced signal in the microscopic image for those cell wall sections located in parallel to the direction of observation during detection of a surface antigen, which emphasizes an annular or oval peripheral line. In contrast to this, conventionally immobilized cells do not show a signal amplification by superposition in the fringe area, but rather an approximately uniform signal over the area, i.e. without signal amplification in the fringe area of the top view. Currently, it is concluded from these observations that the use of the carrier substrate provided with oligonucleic acids for immobilization and analysis of cells according to the invention maintains the spherical cellular shape, whereas conventional carrier substrates flatten the cellular shape.

The contacting of immobilized cells within the channel is possible through a liquid flow along the channel, for example by adding an aqueous composition with a specific detection conjugate, e.g. a dye-coupled antibody at a first opening of the channel and exiting of the aqueous composition through an opposite second opening of the channel, preferably supported by removal of exiting liquid. Due to the small inner volume of the channel according to the invention, for example in the range of 1 to 200 μL, preferably 2 to 20 μL, volumes of 0.5 μL to 200 μL, preferably 1 to 10 μL of the aqueous composition having a content of detection conjugate, e.g. dye-coupled antibodies, are sufficient for feeding the first detection conjugate to the immobilized cells and subsequent to its detection and bleaching feeding a further one.

A first opening of the channel is formed at a first end of the channel, e.g. by the cross-sectional area, which is opened-up by the carrier substrate and the spaced lid as well as by two spacers, whereas in the opposite second end of the channel a second opening is formed, e.g. by the cross-sectional area of the channel, which is opened-up between the carrier substrate and the lid as well as by the spacers arranged between these. Preferably, spacers confining the cross-section of the channel are connected to the lid, whereas the carrier substrate having the coating of oligonucleic acids is arranged against the spacers, optionally only by non-positive arrangement or having a positive fit arrangement and/or with an adhesive applied between the spacers and the carrier substrate. In the analytical method, the carrier substrate is preferably arranged horizontally and above the lid, in particular during the detection such that the surface of the carrier substrate coated with oligonucleotides forms the upper limitation of the channel and e.g. cells immobilized on the carrier substrate are suspended into the channel between the carrier substrate and the lid.

In embodiments in which a frame having positive fit to a tissue sample is arranged at the carrier substrate, the frame preferably has a height of 0.8-fold to 5-fold of the layer thickness of the tissue sample, preferably a height of 1-fold to 1.5-fold of the layer thickness of the tissue sample, wherein the lid is arranged with a spacing to the tissue sample and the frame. Preferably, the surfaces of the frame are arranged perpendicularly to the surface of the carrier substrate coated with oligonucleic acid. In this way, the frame having positive fit is arranged around the tissue sample and enables the passage of aqueous compositions through the channel which is generated between the carrier substrate and the lid so that these, for example washing solutions and antibody conjugates flow into the channel at an inlet opening and can contact the tissue sample, while the aqueous composition preferably exits at an outlet opening opposite the inlet opening, preferably supported by suction.

As an alternative to the immobilization of living cells and/or of tissue the carrier substrate that is provided with a coating of oligonucleic acids and/or with a frame having positive fit to the tissue sample according to the invention can be used for immobilization of denatured and/or of perforated cells and tissue samples. Preferably, in the method according to the invention living cells or tissue sections are immobilized on the carrier substrate that is provided with oligonucleic acids and/or with a frame, which subsequent to analysis, for example after a period of time of 1 to 10 hours, optionally were analyzed additionally in the immobilized state as denatured and/or perforated, for example by adding formalin and/or acetone and/or buffer with saponin (obtainable as FixPerm from BD Biosciences), and/or methanol.

As an alternative to living or dead and non-denatured cells, fixed and/or denatured cells can be immobilized from a suspension on a carrier substrate in the method according to the invention by contacting the suspension with the carrier substrate.

The carrier substrate having the coating consisting of oligonucleic acids and/or having a frame having positive fit to a tissue sample can therefore also be used in analytical methods comprising denaturing and perforating of the immobilized cells such that immobilized living cells subsequent to the analysis in their living state are optionally denatured and/or perforated on the carrier substrate, and are analyzed in the then denatured and/or perforated state in the same position in the visual field of the optical detection device as in their living state.

The coating of the silicate-containing carrier substrate with oligonucleic acids according to the invention is preferably formed of oligonucleic acids bound directly covalently and/or by means of coupling groups and which are arranged at the surface of the carrier substrate in a dense arrangement, preferably in an essentially continuous arrangement, meaning uninterrupted, at least in a detection section of the carrier substrate. The detection section of the carrier substrate is located within the channel and is covered for the optical analysis by the visual field, i.e. by the optical detection range of the optical detection device. Particularly preferred, the carrier substrate which is coated with covalently bound oligonucleic acids forming a continuous coating in the detection section is obtainable by conversion of the carrier substrate at least in the detection section by contacting with oligonucleotides containing alkoxysilane groups in an aprotic phase, e.g. in an organic phase. This is because it has turned out that the contacting of a silicate surface in an organic phase with oligonucleotides containing alkoxysilane groups results in the covalent binding of oligonucleotides to the silicate surface. Preferably, the silicate surface having alkoxysilanes, particularly methoxysilanes, has reactive hydroxyl groups which are for example obtainable by etching as a pretreatment, for example by contacting with aqueous hydrofluoric acid (HF).

Preferably, the analytical method and the production of carrier substrates for the use in the analytical method, respectively, comprises the following steps:

Generating reactive hydroxyl groups on a silicate-containing carrier substrate by contacting the carrier substrate with a strong acid, in particular in aqueous solution, e.g. selected from HF and/or, optionally fuming, sulfuric acid,
exchanging of the strong acid against an organic aprotic solvent, e.g. selected from ether and acetone,
removing of the organic aprotic solvent and contacting of the carrier substrate with oligonucleic acids having an alkoxysilane group at one or at both ends, in alcoholic composition, e.g. in methanol,
washing with an aqueous composition, e.g. water or a cell physiological medium, wherein these steps are carried out without direct contacting of the carrier substrate by atmospheric oxygen and in direct succession, i.e. without drying of the carrier substrate, e.g. by intermediate removal of a liquid film from the carrier substrate. The use for immobilization or for cell analysis, i.e. the contacting of the carrier substrate with cells in an aqueous composition preferably also occurs without direct contacting of the carrier substrate by atmospheric oxygen and in direct succession i.e. without drying of the carrier substrate e.g. by intermediate removal of a liquid film from the carrier substrate.

It has shown that continuation of a liquid film during the afore-mentioned steps for producing the carrier substrate provided with oligonucleic acids and during the contacting with suspended cells and in the subsequent analysis of cells immobilized on the carrier substrate, respectively, the efficiency of the immobilization, e.g. the density of immobilized cells is increased and e.g. supports the natural spherical shape of the cells in their immobilized state and/or the precision of the analysis.

The silicate glass surfaces coated with covalently bound oligonucleic acids according to the invention in the use for immobilization of cells have the advantage that cells are immobilized in a living condition by contacting without further reactive additives in a reproducibly high density.

This reproducible density of the immobilization of cells essentially occurs independent from the binding behavior the silicate glass has without the coating by oligonucleic acids so that by the coating according to the invention a high and reproducible binding effect for cells is generated essentially independent from the binding properties of the uncoated silicate glass.

The oligonucleic acids, in particular the oligonucleic acids connected by an alkoxysilane group, e.g. oligonucleic acids containing methoxysilane groups comprise nucleic acid molecules or deoxyribonucleic acid molecules having at least 5 to at least 50, preferably at least 10 to at least 10,000 nucleotides, which e.g. are synthesized or are DNA and/or RNA isolated from biological material. The alkoxysilane group covalently bound to the oligonucleic acid preferably is a di- or tri-alkoxysilane group and can be connected to the oligonucleic acids via conventional methods of synthesis, e.g. by means of coupling groups which are arranged between a terminal phosphate group of the oligonucleic acid and an alkoxysilane group.

Silicate glass, e.g. borosilicate glass, coated with covalently bound oligonucleic acids according to the invention is obtainable by contacting a silicate-containing carrier substrate having reactive hydroxyl groups under aprotic conditions with oligonucleic acid, which has groups reactive with hydroxyl groups, e.g. in a method containing the steps of:

Contacting a silicate-containing carrier material with hydrofluoric acid, preferably 0.1 to 10 wt.-% in water,

removing the hydrofluoric acid from the carrier substrate,
contacting the carrier substrate with an aprotic, particularly an organic solvent, and
contacting the carrier substrate with oligonucleic acids which contain at least one reactive silicate group, for example oligonucleic acids containing alkoxysilane, in particular oligonucleic acids containing methoxysilane, for example oligonucleic acids conjugated to methoxysilane, in an aprotic, particularly in an organic solvent.

Preferably the alkoxysilane group is a C₁- to C₆-alkoxysilane group, in particular a methoxysilane group or ethoxysilane group. In general, the oligonucleic acid then has for example an alkoxysilane group of the following structure:

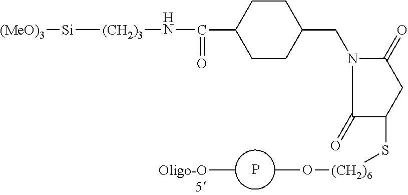

Preferably, the silicate-containing carrier substrate, e.g. silicate glass that is coated with covalently bound oligonucleic acids, during the method of production, during the storage until use for immobilization, and during the immobilization and the analytical method is kept in contact with a liquid at least in the section coated with oligonucleic acids. This is because it was found that the drying of the section of the silicate glass coated with oligonucleic acids leads to an increase of the nonspecific binding of antibody conjugates which are used in the analytical method.

The removal of the hydrofluoric acid from the carrier substrate and the subsequent contacting with organic solvent, which preferably is a C₁- to C₆-alcohol, particularly preferred methanol, ethanol, and/or acetone, can occur by the organic solvent displacing the hydrofluoric acid which preferably is present in aqueous solution.

The coating of the silicate-containing carrier substrate with oligonucleic acids according to the invention on the carrier substrate generates a highly dense, preferably monomolecular layer of oligonucleic acids, having a high capacity for immobilization of cells, particularly for animal cells independent from cell-type and/or independent from surface markers of the cells. Therefore, the oligonucleic acid-coating of the carrier substrate according to the invention allows the non-cell specific immobilization of living cells, particularly of bacterial and of animal cells so that during analysis of a biological sample each cell-type contained therein is immobilized in relation to its proportion in the sample, and the immobilization generates no cell-specific selection. This is particularly advantageous in the analysis of surface markers of cells, because the specific detection of surface markers of cells can be chosen independently from the immobilization of the cells on the carrier substrate, namely only by selection of a marker-specific probe, preferably by selection of the antibody portion of an antibody conjugate having a fluorochrome portion.

In comparison to known methods for the immobilization of cells which are each directed towards the cell-type specific immobilization the surface of the carrier substrate provided with oligonucleic acids and optionally in addition with a frame having positive fit to the tissue sample has an essentially higher capacity for binding living cells; furthermore, the immobilization is stable, for example over a period of time of up to 8 hours for living cells so that a repeated contacting of the immobilized cells by repeated flowing of antibody conjugates in aqueous composition which have differing antigen specificities each, is possible essentially without loss of cells.

An advantage of the silicate-containing carrier substrate coated with oligonucleic acids is that the immobilization can take place without complex sample preparation, e.g. for blood after treatment only by erythrolysis, preferably after separation of lysed erythrocyte components from the intact cells by centrifugation, or after the treatment of samples by isolation of a desired cell-type, e.g. by Ficoll gradient centrifugation, flow cytometry methods or sorting methods using cell-specific magnetic particles (MACS, Miltenyi Biotech). Other medical samples which do not contain impurities or erythrocytes could be immobilized directly without preparation of the sample, e.g. liquor, ascites, bronchio-alveolar lavage, fine needle aspirate, tissue or organ biopsies, optionally after individualization of cells.

Preferably, the carrier substrate for detection is arranged at an optical detection device so that the surface of the carrier substrate that is coated with oligonucleotides is positioned horizontally, preferably above the lid so that immobilized cells are arranged below the carrier substrate. For detection it is preferred that the carrier substrate as well as the lid are transparent.

The method for detection of cell-bound antigens according to the invention is performed by
immobilization of cells, which can be individual cells, cell agglomerates and/or tissue samples, e.g. in suspension, by contacting to the detection section of the carrier substrate coated with oligonucleotides and/or by arrangement within a frame having positive fit which is arranged directly on or at a spacing on the carrier substrate, preferably for use with tissue sections,
contacting the cells by a first antibody conjugate having a first antigen specificity and a fluorochrome portion,
detecting radiation which is emitted by the fluorochrome portion upon irradiation by light having an excitation wavelength,
deactivating or inactivating the fluorochrome portion, for example by irradiating, e.g. by UV light or at the excitation wavelength of the fluorochrome for bleaching,
contacting the cells by a second or a further antibody conjugate having a second or further antigen specificity and the same or a differing fluorochrome portion as the first antibody conjugate, and
detecting the radiation emitted by the fluorochrome portion of the second antibody conjugate upon irradiation by light having an excitation wavelength, optionally with washing of the immobilized cells after contacting with one of the antibody conjugates, for example by flowing of an aqueous composition, for example of a medium for cell culture and/or of buffer through the channel a surface section of which is formed by the carrier substrate,
repetition of the steps of contacting, detecting and deactivating the fluorochrome portion using a further antibody conjugate each time.
Preferably, the method contains the step of automatically determining the position of fluorescence signals detected in relation to the position of the carrier substrate, and
the virtual positionally accurate superimposition of images of fluorescence signals detected, wherein preferably the images which were each detected after contacting with a detection conjugate are presented in a color specific for the detection conjugate, respectively.

As an alternative to the cyclic sequence of contacting immobilized cells with an antibody-dye conjugate, removal of uncombined antibody conjugate, detection of the antibody conjugate and deactivating of the antibody conjugate, the method can comprise the contacting of the immobilized cells with at least two or more antibody conjugates and their simultaneous detection. When using two or more antibody conjugates, these preferably have differing fluorochrome portions and detection takes place for at least two wavelengths, in order to detect differing fluorochrome portions separately from one another.

The process steps of contacting immobilized cells or tissue pieces with antibody conjugates, irradiating with an excitation wavelength, detection of emitted radiation, deactivation of the fluorochrome portion of the antibody conjugate, e.g. by bleaching, can occur repeatedly successively with different antibody conjugates, respectively, which differ in their antibody portion or in their antigen specificity, wherein optionally the fluorochrome portion of the antibody conjugate is the same each. Because the cells are immobilized site-specifically on the carrier substrate in the method according to the invention, the image taken upon the detection of emitted radiation is preferably stored with a site-specific assignment of the image to the carrier substrate. The site-specific assignment of the image can take place by the carrier substrate during detection always having the same position towards the optical detection device, e.g. the same position on the object stage of a microscope. Alternatively, the assignment and storing of the position of the image can take place by identification of the position of a mark on the carrier substrate in relation to the visual field and/or to the beam path of the detection device. Based on the position determined by the immobilization of the cells on the carrier substrate a repeated detection of the same cell or of the same section of the carrier substrate is possible using antibody conjugates having differing antigen specificities each, wherein each antibody conjugate is detected separately, and therefore interfering interactions with other antibody conjugates are reduced or avoided.

As an alternative or in addition to the specific detection of cell components by means of an antibody conjugate the method according to the invention can comprise at least a tissue staining before or after contacting with antibody conjugate, e.g. the hematoxylin-eosin stain and/or the May-Grünwald stain.

Preferably, during the virtual superimposition of images which were detected when contacting the sample with different antibody conjugates, the site-specific assignment of the image, the site-specific assignment of cells is combined with the virtual pattern recognition of a light-microscopic image taken for each image and the virtual corresponding alignment of the recognized pattern of cells for improvement of the precision.

Furthermore, the repeatability of the analysis of living cells using antibody conjugates of differing antigen specificities during a period of at least 5 to 10 h allows the selection of antibody conjugates on the basis of the images which were detected during previous contactings of immobilized cells with antibody conjugates having differing antigen specificities. This iterative analytical method allows the position-dependent assignment of the images detected for each antibody conjugate and their virtual superimposition. Therefore, by the position-dependent assignment of images the analytical method according to the invention on the one hand allows the analysis of the same cells and of the same tissue piece, respectively, and the subsequent virtual superimposition of the images, and thereby an exact analysis of single cells or of tissue portions, and on the other hand the selection of antibody conjugates having a particular antigen specificity on the basis of the images, which were detected previously for a section of the carrier substrate for a selected and determined position for antibody conjugates. The selection of the antibody conjugates can occur without coordination of the fluorochrome portion to fluorochrome portions of other antibody conjugates, since interactions of the fluorochrome portions are avoided, such that the antibody conjugates used subsequently can have the same fluorochrome portion.

Particularly preferred, the method contains the step of photographing a light microscopic image of the cells and an automatic determination of their position, optionally coupled with the step of the positionally accurate coordination of the automatically determined positions to detected fluorescence signals. The position-dependent coordination of the images generated from the detection of fluorescence signals is a positionally accurate repeatable detection of a section of the carrier substrate or of the cells immobilized thereon, especially in embodiments in which the carrier substrate upon detection of different antibody conjugates assumes the same position in the visual field of the optical detection device.

In embodiments of the analytical method which contain the step of virtual superimposition of positionally accurate images of a section of the carrier substrate having living prokaryotic and/or eukaryotic cells immobilized thereon, the method is not limited to the immobilization on carrier substrates coated with oligonucleic acids, wherein the images are each generated by repetition of the steps of contacting the cells with antibody conjugate, the detection of the antibody conjugates contacted with the cells and the inactivation of the fluorochrome portion of the antibody conjugates with antibody conjugates of different antigen specificity in each step. As an alternative to coating of the carrier substrate with oligonucleic acids which is especially preferred for use for the immobilization of living animal cells, the carrier substrate can also have another conventional coating, e.g. with polylysin, or can consist of a synthetic material which promotes the adhesion of cells, e.g. polystyrene. For the method of analysis of cells which are denatured, the fixation on a carrier substrate can occur without specific coating.

### DETAILED DESCRIPTION OF THE INVENTION

The invention will now be described in greater detail by way of examples with reference to the Figures, in which

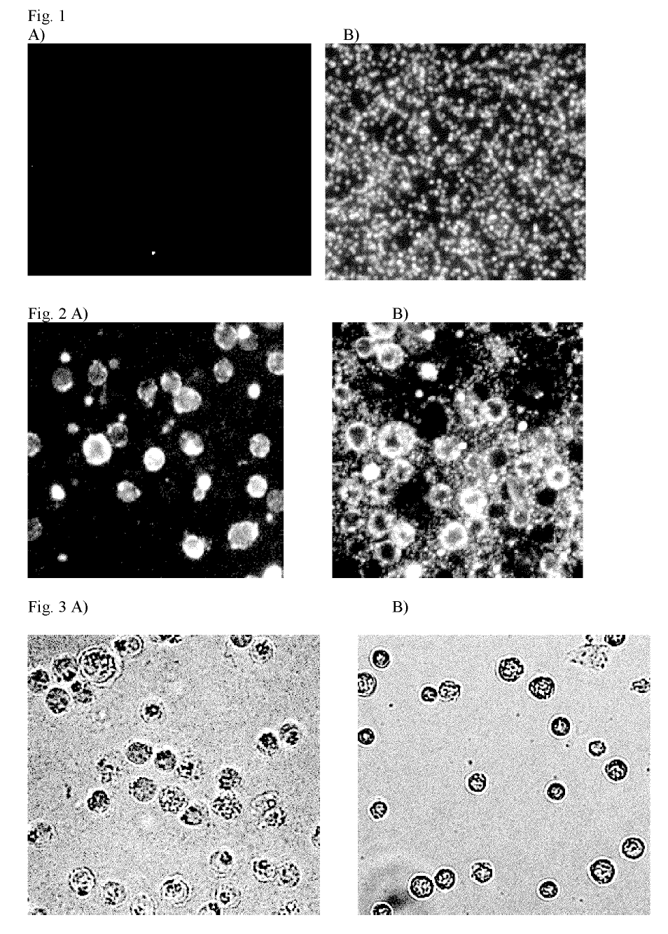

FIG. 1 under A) shows a purified glass surface for comparative purposes, and under B) shows a glass surface coated with oligonucleic acids in single molecule detection,

FIG. 2 shows the images of immobilized cells following antigen-specific labelling with fluorescence detection under A) for comparison on a purified glass surface, and under B) on a glass surface coated with oligonucleic acids,

FIG. 3 shows light microscopic images of cells, under A) for comparison on a purified glass surface, and under B) on a glass surface coated with oligonucleic acids,

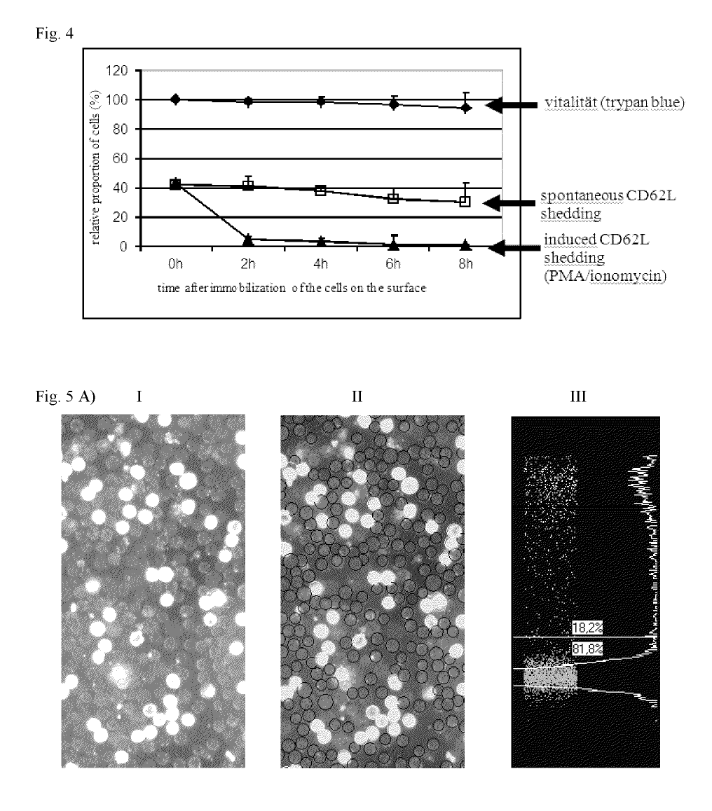

FIG. 4 shows the results of a vitality test of living human immune cells immobilized on a carrier surface with and without nonspecific stimulation,

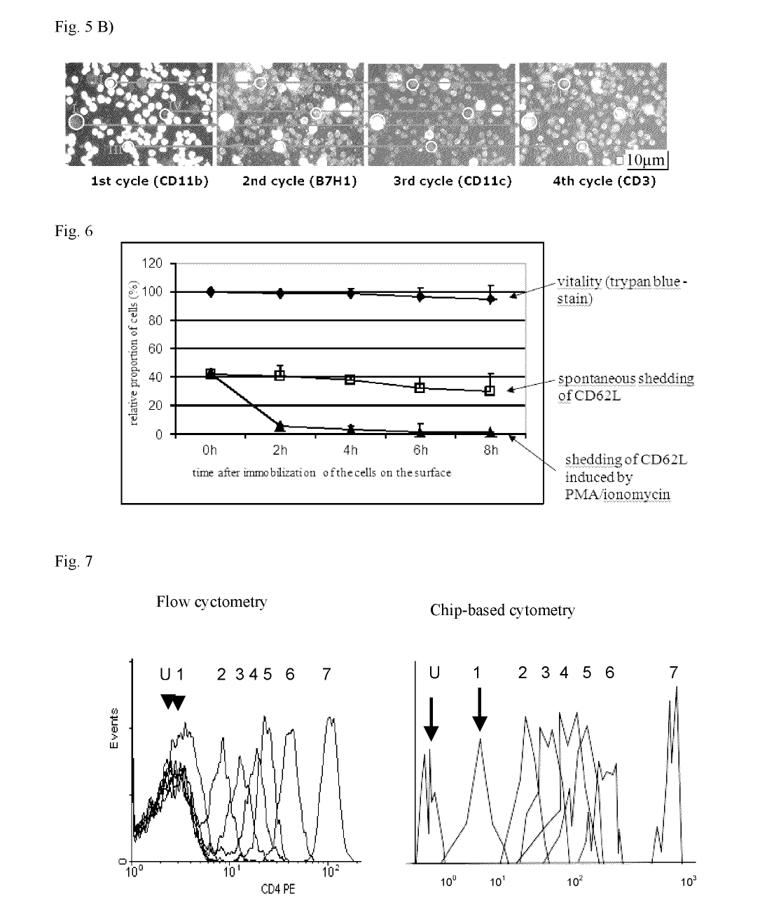

FIG. 5 A shows under I) immobilized leukocytes of the mouse after incubation with anti-CD 4-antibody-PE-conjugate in a microscopic image under excitation of the fluorescence, under II) the automatic recognition and intensity measurement of the radiation emitted by the fluorochrome, and under III) a graphic representation of the fluorescence intensity (Y-axis) over the number of cells (X-axis) in the form of a dot-plot and histogram,

FIG. 5 B) from left to right shows microscopic images under fluorescence excitation of subsequent incubations, each with antibody conjugates of different antigen specificity in the method according to the invention,

FIG. 6 shows images of the detected emission of fluorochromes of subsequently used antibody conjugates with different antigen specificities (as indicated to the left of the images) for subsequently performed contactings each of an immobilized sample (cell 1 and cell 2, respectively), including detection of intracellular antigens with intermittent inactivation of the fluorochrome portion each,

FIG. 7 for aliquots of the same sample with the same antibody conjugates under A) shows the result of an analysis by flow cytometry, and under B) the results of a method according to the invention (iSBC) with human cells as the immobilized biological sample,

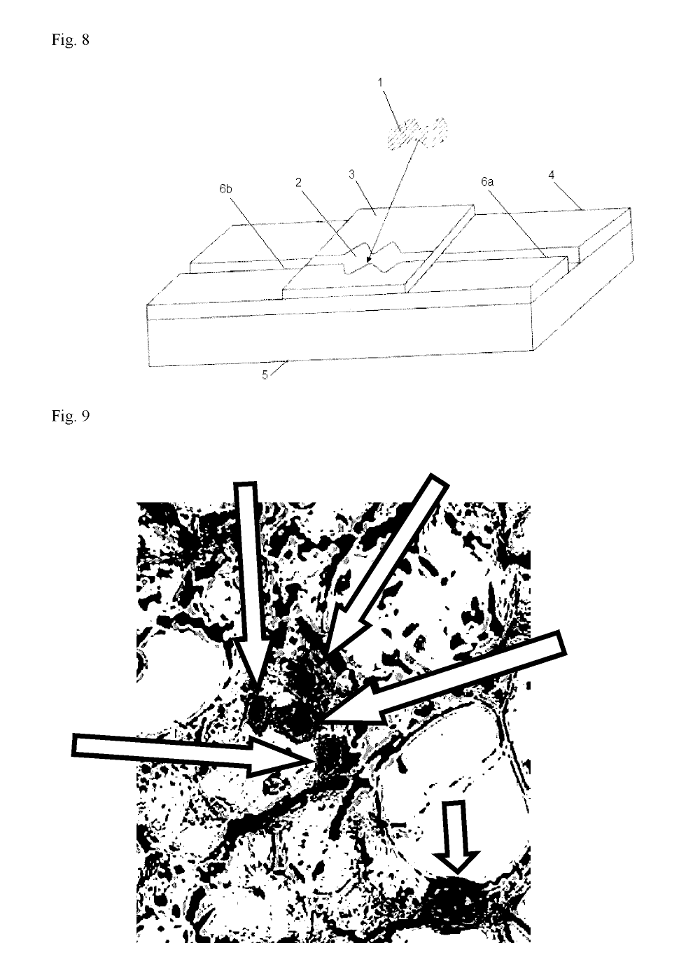

FIG. 8 shows a schematic structure of an embodiment of the carrier substrate coated with oligonucleic acids for analysis of a tissue sample, and

FIG. 9 shows the result of the analysis of a tissue section.

### EXAMPLE 1

### Production of a Flow Channel Having an Oligonucleotide Coating

A channel according to the invention for use in the analytical method of the invention was produced by sectional production of a covalently bound oligonucleotide layer on a carrier substrate containing silicate glass. A conventional cover glass which was on two spaced-apart spacers served as carrier substrate. The spacers were fixed on a microscope slide of glass, serving as a lid. In the channel that was opened up by the carrier substrate, the spacers and the lid, the covalently bound coating of oligonucleotides was generated on the carrier substrate by superficial rinsing or immersing in 1% hydrofluoric acid in water for 10 minutes at room temperature, removal of the hydrofluoric acid by rinsing with acetone, removal of the acetone and contacting with a methanolic solution of oligonucleic acids (dT₃₅) containing tri-methoxysilane groups. Excess oligonucleotide could be removed by rinsing in PBS (phosphate buffered saline, pH 7.4). The tri-methoxysilane group was coupled to the 35-dT-oligonucleic acid (Oligo) in the following compound:

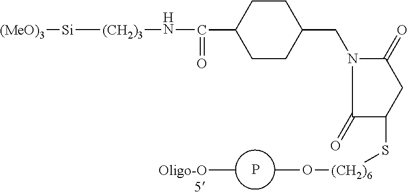

Correspondingly, the Oligo in the place of the 35-dT-mer, another oligonucleotide of synthetic or natural origin can be bound to this or another coupling group by an alkoxysilane group. As a test for the distribution and bonding of the oligonucleic acid molecules to the glass surface of the carrier substrate, a glass surface etched with hydrofluoric acid for comparison to oligo-dT-nucleic acid of the invention was contacted with a fluorescent dye-labelled dA₂₆-Cy3 in high dilution. The result of the fluorescence-microscopic analysis (Axioplan 2e microscope, 100× Plan-Achromat object lens for single molecule detection) is shown in FIG. 1, under A) the purified glass surface for comparison, under B) the glass surface coated with covalently bound dT₃₅. This analysis shows that the fluorescence labelling is a result of the covalently bound oligonucleic acid coating, and the oligonucleic acid molecules are evenly distributed over the surface.

The nucleic acid coating was so complete and continuous, respectively, that the contacted surface upon contacting with a cell suspension, e.g. by Ficoll gradient isolated leukocytes, the cells were immobilized to a single layer cell layer, wherein the density of the immobilized cells was dependent on the number of cells. For single cell analysis it is preferred that the cells are immobilized on the carrier substrate at a spacing, for easier recognition of the single cells by their position relative to the carrier substrate.

The immobilization of cells on the oligonucleic acid-coated silicate-containing carrier substrate takes place by contacting the oligonucleic acid-coated silicate-containing carrier substrate with the cells, which are present e.g. in suspension. For contacting, the deposition of the cells in aqueous composition onto the carrier substrate at 20 to 36° C. is sufficient, e.g. by pipetting of the cell suspension into the inlet opening of the channel comprising the coating of the carrier substrate.

For optical analysis, the microscope slide was placed on the object stage of a microscope such that the cover glass coated with oligonucleic acids was arranged above the microscope slide. Therefore, the carrier substrate coated with oligonucleic acids was arranged above the lid formed by the microscope slide, and above the channel volume.

The distribution of the immobilized cells on the cover glass coated with oligonucleic acids shows that the coating with oligonucleic acids generally results in an even immobilization of cells. The immobilization capacity of approximately 2,000 to 2,500 cells/0.15 μm² (visual field) is significantly higher than that of carrier substrates with immobilized capture antibody. Furthermore, the smaller channel volume above the detection section of about 2 μL allows the use for small volumes of sample and of antibody conjugate. Accordingly, for an inner volume of the channel bordering on the detection section coated with oligonucleic acids approximately 10,000 to 100,000 cells in a volume of about 2 μL are sufficient for obtaining a density of immobilized cells of approximately 400 to 1800 cells/0.15 μm², which is currently regarded as optimal.

Further, a comparison of the analysis of an aliquot of cells which was contacted with a glass surface coated with oligonucleic acids to an aliquot that was contacted with a glass surface purified by etching in hydrofluoric acid shows that the coating with oligonucleic acids according to the invention reduces nonspecific signals in fluorescence detection, and allows a more sensitive detection of fluorescently labelled analytes, respectively. Fluorescence-microscopic images are shown in FIG. 2A) for the purified glass surface, and in 2B) for the glass surface coated with dT₃₅ after contacting with human PBMC (mononuclear cells of blood), and contacting with 10 μg/mL anti-CXCR5-PE-antibody conjugate for 5 min. It becomes clear that the coating with oligonucleic acids results in a significantly improved detection of the specifically labelled cells, whereas nonspecific background signals are significantly reduced by the coating with oligonucleic acid.

The light microscopic analysis shows the specific suitability of the coating with oligonucleic acid for use in the fixation of permeabilized eukaryotic cells. In FIG. 3A), PBMC are shown on the glass surface purified by etching with hydrofluoric acid, in 3B), the PBMC are on the glass surface coated with dT₃₅, wherein the cells after contacting the surface have been fixed and permeabilized by the saponin-containing buffer (FixPerm, BD Biosciences) each. The contours of the cells on the glass surface coated with oligonucleic acid clearly are better maintained than the contours of the cells on the purified glass surface. This shows that the coating with oligonucleic acids better preserves the cell structures upon fixation and permeabilization than a non-coated surface.

Upon the analysis of the glass surface provided with oligonucleic acids it has turned out that a reduction of the concentration of the dT35-methoxysilane-oligomer used in the production process results in a not sufficiently dense binding of oligonucleic acids. Such it was found by single molecule detection of dA26-Cy3 to immobilized oligo-dT35 that at concentrations of 0.0017 pm/4 dT35-methoxysilane-oligomer, 14 molecules oligo-dT35/100 μm² of the glass surface, at 0.017 pm/μL dT35-methoxysilane oligomer 187 molecules oligo-dT35/100 μm² of the glass surface, and at 0.17 pm/4 dT35-methoxysilane oligomer >5,000 molecules oligo-dT35/100 μm² of the glass surface were bound, which corresponds to or exceeds the resolution of the microscope at the fluorescence detection used. For an efficient and cell-type—nonspecific binding of cells to the carrier substrate provided with oligonucleic acids, this preferably in general has a density of at least 5,000 molecules oligonucleic acids per 100 μm² surface, preferably a density of at least 10,000 molecules oligonucleic acids per 100 μm² surface.

### EXAMPLE 2

### Immobilization and Analysis of Living Eukaryotic Cells

The analytical method according to the invention is suitable both for immobilized living cells and for immobilized cells, which after contacting with the silicate surface coated with oligonucleic acids are denatured conventionally, and/or are perforated, e.g. by incubation with formalin, acetone, methanol and/or saponin.

As an example for eukaryotic cells, living human immune cells were used, which after removal of erythrocytes were present in suspension in PBS. For immobilizing the cells on the carrier substrate coated with oligonucleotides, these were flowing into the channel, one inner side of which was formed by the carrier substrate coated with oligonucleic acids.

FIG. 4 shows the functional analysis of living human immune cells which are immobilized on the surface of a cover glass coated with oligonucleic acids according to Example 1. The vitality stain with 10 μL trypane blue shows that at least for an immobilization for a duration of 8 hours, the number of living cells decreases only slightly. The shedding of CD62L from the cell surface (open boxes) without addition of a stimulant, e.g. spontaneously, at least over 8 hours, preferably over 2 to 4 hours shows an only slight impairment of the cells by the immobilization, in contrast to activation by addition of PMA/ionomycin, which resulted in a shedding of CD62L within the first two hours of fixation.

Accordingly, it is preferred to first carry out the method for analysis on living immobilized biological samples, especially on eukaryotic cells, and to subsequently denature and/or perforate these cells, for subsequently performing the analytical method on the denatured and fixed cells, respectively.

### EXAMPLE 3

### Detection of Surface Antigens on Immobilized Living Eukaryotic Cells with Automatic Determination of the Position of the Cell

Leukocytes from peripheral blood of the mouse after erythrolysis were separated by centrifugation from remainders of the erythrocytes and pipetted in serum onto a carrier substrate that was produced according to Example 1, which was covered by a microscope slide as a lid at a spacing of 20 μm. After incubation at room temperature for 5 min in the horizontal, the supernatant was displaced by addition of PBS (pH 7.4).

The carrier substrate was positioned in the beam path of a microscope (Axioplan 2e microscope, Zeiss, with motorized adjustment of the object stage and focusing, mercury vapour lamp HBO100 for excitation, filter for PE or FITC, immersion objective Plan-Neofluar 16×/0.50, and a CCD-camera Axiocam MRm for recording, used in all Examples) following or prior to addition of PE-conjugated anti-CD4-antibodies (10 μg/mL, 10 μL) in PBS. In correspondence to the preferred embodiment, the cover glass used as carrier substrate was arranged horizontally and above the microscope slide (lid) within the visual field of the microscope, such that the detection section coated with oligonucleic acids formed the upper wall of the channel between the carrier substrate and the spaced lid. In this position, the cells hang into the inner volume of the channel and it was found that in this way the immobilization only marginally influences the morphology of living cells.

After incubation at room temperature for 5 min, uncombined antibody was removed by washing of the immobilized cells by addition of 100 μl, PBS at one end of the cover glass, and suction of the exiting liquid at the opposite end. The detection of bound antibody was done microscopically by irradiation with a wavelength of 488 nm. The microscopic image with fluorescence excitation is shown in FIG. 5A) I, the automatic image recognition that is preferably performed is shown in II, and the assignment of each cell to a certain position of the detection section done by the automatic image recognition in connection with the automatically determined position of the carrier substrate is shown in III, allowing a defined assignment of each cell to a spatial position relative to the carrier substrate, and therefore allows an unequivocal identification and assignment of each cell without shifting of the carrier substrate, and also after removal and renewed arrangement of the carrier substrate within the visual field of the microscope on the basis of the automatically determined position on the carrier substrate.

The automatic determination of the position of cells in the visual field of the microscope took place by means of a self-developed program with additional automatic assignment of the position of the computer-controlled moveable object stage (Merzheuser, Germany) to every position determined for a cell. The images were taken by detection for a duration of 7 s with fluorescence detection (transmissive light 100 ms). Upon removal of the carrier substrate from the object stage, e.g. for further addition and/or removal of solutions for washing and with further antibodies, the position of the cells on the carrier substrate was not altered, so that only by correlating of the positioning of the carrier substrate and of the object stage supporting this in the same position, each cell could be detected anew positionally accurate, and microscopic fluorescence images taken subsequently for each cell with different antibodies could be superimposed specifically. The circles introduced into FIG. 5 A) II stand for positions of cells not labelled by antibody, which were identified in a previous light microscopic image. FIG. III shows the distribution of the intensity of fluorescence measured for all cells over the number of cells.

### EXAMPLE 4

### Subsequent Detection of Different Surface Antigens in Immobilized Living Eukaryotic Cells

Cells from a bronchio-alveolar lavage of a mouse with induced asthma (according to Polte et al., J. Allergy Clin. Immunol. 118, 942-8 (2006)) were immobilized according to Example 2. The immobilized cells were contacted subsequently with antibody conjugates, each containing the fluorochrome PE, each with inactivating the fluorochrome prior to addition of a new antibody conjugate, e.g. by irradiation at the excitation wavelength specific for the fluorochrome (488 nm) for approximately 30 s, with washing after addition of a new antibody and detection under fluorescence excitation. The antibody conjugates only differed in their antibody portion, which had a specificity for CD11b, B7H1, CD11c, and CD3, respectively.

FIG. 5B), arranged from left to right, shows microscopic images with detection of emitted fluorescence; the specificities of the antibodies, namely anti-CD11b, anti-B7H1, anti-CD11c, and anti-CD3, are indicated below the respective images. The graphic labelling of single locations shows that the assignment according to the invention of the fluorescence specifically detected for an antibody conjugate each to the position of the microscopic image allows the superimposition of subsequently detected fluorescence signals. As a consequence, the method of the invention allows the detection of several antigens to be analyzed in immobilized biological samples subsequently, each with antibody conjugates having specificity for the antigens to be analyzed, with the same fluorochrome portion or with different fluorochrome portions. This analysis shows that single positions of a biological sample, e.g. single cells, can be analyzed subsequently for a majority of antigens without impairments of the analysis by interactions of antibody conjugates or by their fluorochromes occurring, or without nonspecific fluorescence being generated, wherein the surface of the carrier substrate coated with oligonucleic acid according to the invention allows a sufficiently extended immobilization of the cells in living condition. Based on the immobilization, the automatic determination of the position of each cell analyzed in light microscopy and of the detected fluorescence signals is possible, and the subsequent positionally accurate superimposition or correlation of the microscopic image and/or of the detected fluorescence signals.

The superimposition of the same positions of detected fluorescence each, preferably in combination with the light microscopically determined positions of cells allows the subsequent detection of different antigens in separate steps and their subsequent assignment to each cell. This positionally accurate correlation of the detected fluorescence signals is presented in FIG. 5B) by the added connection lines between encircled cells.

### EXAMPLE 5

### Subsequent Detection of Intracellular and Surface Antigens in Fixed and Permeabilized Eukaryotic Cells

As an example for a biological sample, human leukocytes were immobilized according to Example 2, but subsequently fixed with formaldehyde and permeabilized with buffer containing saponin (FixPerm, BD Biosciences).

The cells fixed on the carrier substrate coated with oligonucleic acids were contacted with 10 μg (2 μg/mL-200 ng/mL) antibody conjugate for 5 min at room temperature and washed with 200 μL PBS. Generally, after the contacting with an antibody conjugate, the radiation emitted upon irradiation with light of the excitation wavelength was detected, subsequently the fluorochrome portion of the antibody conjugate was deactivated by irradiation at a wavelength specific for the fluorochrome (488 nm), and subsequently another antibody conjugate was contacted to the sample.

The cover glass serving as a carrier substrate that was coated with oligonucleic acids was arranged above a microscope slide, which was spaced by two bridge-shaped spacers of about 150 μm thickness from the carrier substrate, such that the bridges limited the room between the carrier substrate and the spaced microscope slide to a flow channel. The steps of contacting the sample with antibody conjugate and the washing steps after addition of another antibody conjugate could each be performed by pipetting of the respective aqueous composition to one end of the flow channel, optionally in combination with suction of liquid at the opposite end of the flow channel.

After incubation of the immobilized cells with the anti-IFN-γ-antibody conjugate for 5 min at room temperature, and a subsequent washing step, fluorescence was detected microscopically. The detected fluorescence including the microscopic image were recorded with assignment to the positioning of the carrier substrate, and with unchanged positioning of the carrier substrate, respectively, by storing in an electronic storage. Subsequently, the fluorochrome portion was bleached by irradiation at 488 nm.

Subsequent to this inactivation of the fluorochrome portion of the first antibody conjugate having specificity for IFN-γ, an antibody conjugate was contacted to the sample that was constructed identically, except that the antibody portion now was an anti-CD3-antibody. Again, the fluorochrome portion of the antibody conjugate having anti-CD3-specificity was deactivated by irradiation at 488 nm subsequently.

In the same manner, the same sample was subsequently incubated with antibody conjugates having an anti-CD4-antibody portion, an anti-CD8-antibody portion, and an anti-IL4-antibody portion, respectively, each with inactivation of the fluorochrome portion prior to incubation with an antibody having a different (additional) antigen specificity, each with a washing step following the addition of an antibody conjugate for removal of unbound fluorochrome.

Microscopic images are shown in FIG. 6 for cell 1 and for cell 2, wherein essentially the same image section was detected, which is marked by the manually introduced circle. It is clear that the inactivation of the fluorochrome portion of the respective antibody conjugate results in a reduction and elimination, respectively, of emissions from the previously used antibody conjugate, at least to a level below the detection limit, wherein also in further cycles of the analytical method with antibody conjugates of different antigen specificity no additional background activity or background fluorescence occurred. Further, this example shows that both the extracellular antigens CD3, CD4, CD8, and the intracellular antigens IFN-γ and IL4 can be detected independent from one another in the fixed cells.

### EXAMPLE 6

### Comparison of the Specificity and of the Sensitivity of the Process According to the Invention in Respect to Flow-Cytometry

The analytical method of the invention was analyzed on human leukocytes which were immobilized on a carrier substrate and for comparison by conventional flow-cytometric analysis. Aliquots of spleen cells of the mouse (5 animals) were contacted with antibody conjugates of different antigen specificity, to test the identification of the respective antigens on the cells in comparison. For the comparative analysis using flow-cytometry (FACScan, Becton Dickinson), aliquots of the cell suspensions in 50 μL PBS with 1% RSA were incubated with one of the following antibody conjugates (1 μL, 0.2 μg/mL) each: Anti-CD3-PerCP, anti-CD4-PE, anti-CD8-FITC, anti-CD19-PerCP, anti-CD4-PE, anti-CD3-FITC, respectively, and analyzed separately for antibody conjugates with the same fluorochrome portion.

For the analytical method of the invention, leukocytes were immobilized on a carrier substrate coated with oligonucleotides which was arranged in a flow channel and contacted with the following antibody conjugates one after another, each with intermediate inactivation of the fluorochrome portion by UV irradiation: anti-CD3-PE, anti-CD4-PE, anti-CD8-PE, anti-CD19-PE, also in 50 μL PBS with 1% RSA and 1 μL, 0.20 μg/mL antibody conjugate.

The following proportions of cells were detected with the antibody conjugates:

| | | In accordance with |
|---|---|---|
| | | the invention on |
| antigen | flow cytometry | immobilized cells |
| CD19+ | 60% +/−1 | 60% +/−0.7 |
| CD19+ CD4+ | 0% | 0% |
| CD3+ | 39% +/−2.5 | 40% +/−3 |
| CD4+ | 24.5% +/−2 | 27% +/−1.5 |
| CD8+ | 15% +/−2 | 14.5% +/−1.5 |
| CD4+ CD3+ | 17% +/−3 | 19% +/−2 |

These results show that the same antibody-specific cell populations were identified in the samples by both methods. Accordingly, the method of the invention essentially has the same specificity as the analysis using flow-cytometry (FACS).

The detection sensitivity of the method of the invention was also tested in comparison to FACS, by immobilizing spleen cells of the mouse on a cover glass coated with oligonucleotides according to Example 2, and contacting one after another with decreasing dilutions, i.e. with increasing concentrations of anti-CD4-PE antibody conjugate, with inactivation of the fluorochrome PE after each detection. For the comparative analysis via FACS, 7 aliquots of the cell suspension with the same dilutions of the anti-CD4-PE were incubated and analyzed.

The results are shown in summary graphically in FIG. 7 A) for flow-cytometry, and in 7 B) for the analytical method of the invention, wherein peak values are marked with indications of the concentration of the antibody with U=negative control without antibody conjugate, and with 1 to 7 for increasing concentrations. The results show that the sensitivity of the analytical method of the invention is higher by about a factor of 10 than for flow-cytometry. Therein, at 1, the lowest antibody concentration, no resolution from background (U) is found in flow-cytometry, whereas the method of the invention already for this antibody concentration generates a detectable signal which is clearly distinguishable from background.

### EXAMPLE 7

### Detection of Macrophages in Lung Tissue

As an example for the detection of cell-specific surface antigens in a piece of tissue with living cells, a tissue section of lung tissue of the mouse using a vibrating microtome (available from the company Vibratome) was immobilized on a carrier substrate. The lung tissue was alive and was immobilized by the carrier substrate having a frame of elastic material having positive fit, by dissecting from a plastic foil a form corresponding to the tissue section and arranging this frame on a cover glass. As plastic foil, polyethylene, polypropylene, and preferably Parafilm could be used. The plastic foil preferably had a thickness of about 200 μm; the tissue section had a layer thickness of about 100 μm. Preferably, the cover glass used as the carrier substrate in the section that was encircled by the frame was additionally coated with oligonucleic acids in accordance with Example 1. As a lid, a microscope slide of glass was arranged opposite the cover glass. The frame with form fit to the tissue piece formed the spacers between the cover glass and the microscope slide, wherein the frame had two opposed openings of strip-shaped recesses in the material of the frame, which oppositely discharge into the volume between carrier substrate (cover glass) and lid (microscope slide) that is surrounded by the frame. The strip-shaped recesses in the material of the frame were formed in the spacers, and provided the inlet opening and the opposite outlet opening in the volume surrounded by the frame, and, accordingly, the inlet and the outlet openings to the tissue piece arranged therein.

A schematic view of the frame having positive fit to the tissue piece is shown in FIG. 8:

The tissue piece 1, e.g. a tissue section, is arranged in a frame 2 having positive fit. The frame 2 having positive fit is arranged between a carrier substrate 3, which can e.g. be a cover glass, and a lid 5, which can e.g. be a microscope slide. The carrier substrate at least in the section which is comprised by frame 2 is preferably coated with covalently bound oligonucleotides. The frame 2 is formed as a recess by spacers 4, which are arranged between carrier substrate 3 and lid 5, wherein carrier substrate 3 and lid 5 limit the volume opened up by the frame 2. Within the spacers 4, strip-shaped or groove-shaped recesses 6a, 6b are formed, which form an inlet opening and an approximately opposite outlet opening within the volume opened up by the frame 2 between carrier substrate 3 and lid 5.

A microscopic image of the tissue section analyzed using anti-mouse-CD11b-antibody-PE-conjugate in accordance with Example 2 is shown in FIG. 9. The cells marked with arrows show the identified CD11b+-cells in the tissue and make it clear that the frame having form fit according to the invention to the tissue piece between the carrier substrate and the lid is suitable for immobilization of living tissue and for analysis of cell-specific antigens in the tissue piece.

The fluorochrome portion PE was bleached by irradiation at 488 nm. Subsequently, in successive cycles, further 30 antibody conjugates could be contacted to the tissue piece. The antibody conjugates had PE as their fluorochrome portion, and a different antibody portion each. For each detection, the carrier substrate was arranged in the same position in the beam path of the microscope used as detection device (Zeiss Axioplan 2e of Example 3). Each cycle comprised the contacting of the tissue piece with an antibody conjugate, washing for removal of uncombined antibody conjugate, detection of the light emitted by the fluorochrome portion under irradiation with light of excitation wavelength (488 nm), storage of the detected image, and the inactivation of the fluorochrome, e.g. by bleaching by irradiation at the excitation wavelength. For evaluation, the images were called from the storage and were superimposed virtually with accurate position, as the single images of each antibody conjugate were detected with identical positioning of the carrier substrate in the detection device. These process steps of the analysis of cells in a tissue piece are the generally preferred method.

1. Method for analysis of eukaryotic cells with the steps of contacting cells or tissue pieces, having a section of a carrier substrate which is arranged within a channel having an inlet opening and an outlet opening,
contacting the cells arranged on the section of the carrier substrate with a first detection conjugate having a first antigen specificity and a fluorochrome portion,
irradiating of light having an excitation wavelength for the fluorochrome portion,
detecting the radiation emitted from the fluorochrome portion,
storing of the image of the detected radiation,
characterized in that
the section of the carrier substrate is coated with oligonucleic acids which in an aqueous medium have a linear conformation and are bound to the carrier substrate with exactly one of their ends.

2. Method according to claim 1, characterized in that the oligonucleic acids are present to at least 5,000 molecules per μm² in the section of the carrier substrate.

3. Method according to claim 1, characterized in that the oligonucleic acids are single-stranded and in at least 90% of the total number of nucleotides have the same base.

4. Method according to claim 1, characterized in that the oligonucleic acids are double-stranded and to at least 90% of the total number of nucleotides consist of adjacent sections, wherein each section consists of identical nucleotides each, the bases of which comprise one of A or T or U or I.

5. Method according to claim 1, characterized in that the oligonucleic acids are bound to the carrier substrate by coupling groups.

6. Method according to claim 1, characterized in that the carrier substrate in addition to the coating with oligonucleic acids has a frame having positive fit to a tissue sample, which frame extends to a lid spaced from the carrier substrate, wherein a tissue piece is arranged within the frame.

7. Method according to claim 1, characterized in that subsequent to the detection of the radiation emitted from the fluorochrome portion, the inactivation of the optical activity of the fluorochrome portion occurs, and
the contacting of the cells arranged on the section of the carrier substrate with a further detection conjugate having a further antigen specificity and a fluorochrome portion,
the irradiation of light having an excitation wavelength for the fluorochrome portion,
the detection of the radiation emitted from the fluorochrome portion,
the storing of the image of the radiation detected each time, and the inactivation of the fluorochrome portion of the further detection conjugate occurs repeatedly,
wherein images which are stored for one contacting with a detection conjugate each are displayed positionally accurate virtually superimposed.

8. Method according to claim 7, characterized in that the inactivation of the fluorochrome portion occurs by irradiation.

9. Process according to claim 7, characterized in that the fluorochrome portions of the antibody conjugates are identical.

10. Use of a silicate-containing carrier material for immobilizing cells with analysis of the cells, characterized in that the cells are nonspecifically immobilized by the carrier material having oligonucleic acids which have a linear conformation and which are bound with exactly one end to the carrier material, and the cells immobilized on the carrier substrate are specifically analyzed by contacting with a detection conjugate which has a binding portion specific for a component of at least one cell, and a fluorochrome.

11. Use according to claim 10, characterized in that the oligonucleic acids are single-stranded and have the same base in least 90% of the total number of nucleotides.

12. Use according to claim 10, characterized in that the oligonucleic acids are double-stranded and in at least 90% of the total number of nucleotides consist of adjacent sections, wherein each section consists of identical nucleotides, respectively, the bases of which each comprise A or T.

13. Use according to claim 10, characterized in that the oligonucleic acids of linear conformation are bound to the carrier material by means of a coupling group, which is bound to exactly one of their ends.

14. Use according to claim 13, characterized that the coupling group is a nucleotide which is bound to the carrier substrate, to which nucleotide two oligonucleic acids of linear conformation are bound.

15. Device for the optical analysis of cells immobilized on a silicate-containing carrier substrate, wherein the device has a channel having an inlet and an outlet opening for liquid compositions, which is formed by the carrier substrate, a lid spaced therefrom, and spacers arranged between carrier substrate and lid, characterized in that a section of the carrier substrate is coated with oligonucleic acids which in an aqueous medium have a linear conformation and which are bound to the carrier substrate with exactly one of their ends.

16. Device according to claim 15, characterized in that the oligonucleic acids are single-stranded and in at least 90% of the total number of nucleotides have the same base or are double-stranded and to at least 90% of the total number of nucleotides consist of adjacent sections, wherein each section consists of identical nucleotides each, the bases of which comprise A or T, respectively.

17. Device according to claim 15, characterized in that the device has a recording unit for recording microscopic images of the carrier substrate, an electronic storage for storing the recorded microscopic images, a calculating unit disposed for positionally accurate superimposition of at least two recorded microscopic images, and a display unit disposed for displaying the positionally accurate superimposed microscopic images.

18. Device according to claim 15, characterized in that the oligonucleic acids are present on the section of the carrier substrate to at least 5,000 molecules per μm² carrier substrate.
